(19) United States
(12) Patent Application Publication
(10) Pub. No.: US 2006/0041275 A1
(43) Pub. Date: Feb. 23, 2006

(54) Infant clothing and accessories with permanent teething element

(76) Inventor: Jill Powers, Federal Way, WA (US)

Correspondence Address: JILL POWERS, PO BOX 3412, KENT, WA, 98089, US

(21) Appl. No.: 11/209,892

(22) Filed: Aug. 23, 2005

Related U.S. Application Data
(60) Provisional application No. 60/603,654, filed on Aug. 23, 2004.

(51) Int. Cl. A61B 17/08

(52) U.S. Cl. 606/215

(57) ABSTRACT

For close to two years of a child's life, children need easy access to teething items. The problem with most teething apparatus on the market is that they are easily misplaced; they fall onto unsanitary surfaces and therefore need to be cleaned often. The present invention solves these problems by providing a simple and effective way for babies to have easy and consistent access to a teething apparatus. A teething strip, usually of non-toxic plastic, is attached by mechanical means (sewn, snapped or riveted) to the baby's clothing within reach of the baby's mouth. When and if the infant drops the teething element, the element does not fall to the floor or otherwise out of the infant's reach. By permanently attaching a teething strip to children's clothing, this invention has the advantages of allowing easy and consistent access to soothe teething pain while at the same time preventing the loss of the teething unit, limiting the exposure of it being dropped onto unsanitary surfaces, eliminating the need to purchase additional teething items and providing a unique design that enhances any outfit.

### CROSS-REFERENCE TO RELATED APPLICATIONS

This application claims the benefit of PROVISIONAL PATENT APPLICATION No. 60/603,654, filed Aug. 23, 2004.

### FIELD OF INVENTION

This invention pertains generally to baby accessories and more specifically to a teething strip that is permanently attached to clothing and baby accessories.

### BACKGROUND

Infants universally enjoy sucking and chewing on various “oral elements.” An artificial nipple, commonly termed a “pacifier,” is a primary example. Another example is the large class of “teething” products designed to provide an infant relief from the pain and itching of cutting teeth.

Pacifiers and teething elements come in a wide variety of sizes and shapes. However, virtually all pacifiers and teething elements are manufactured and sold as small, individual items. The problem with this is that, when the infant drops the element, the infant is unable to locate and re-grasp the element. This is frustrating for the infant, and it is also frustrating to the infant's caregiver who must go to the infant, relocate the oral element, clean it, then re-position the oral element into the infant's hand or mouth.

Another problem with the small individual pacifiers and teething elements of the prior art is that whenever the infant drops the pacifier or teething element, it frequently falls onto the floor, ground or other unsanitary surface.

The same problem applies to the prior art of a teething apparatus being attached to a blanket or towel. Blankets and towels also fall to the floor. They are often placed on or near the floor especially when used with an infant carrier. In addition, blankets are not practical in warm weather.

Attempts have been made to alleviate these problems by attaching the oral element to the infant via some form of tether. Although such tethers tend to prevent the oral element from falling onto an unsanitary surface, they do little to assist the infant in relocating and re-grabbing the device and offer a potential hazard for hands and fingers to be caught in a strap.

Accordingly, there is a need for a simple and inexpensive infant product having a teething element that avoids the above-described problems with the present state of the art

### OBJECTS AND ADVANTAGES

The present invention solves the above-mentioned problem by providing a simple and effective way for babies to have easy and consistent access to a teething apparatus. Among the several advantages are:
(a) A teething strip, usually made of non-toxic plastic, is permanently attached by mechanical means (sewn, snapped or riveted) to the baby's clothing or accessories within reach of the baby's mouth.
(b) When and if the infant drops the teething element, the element does not fall to the floor or otherwise out of the infant's reach.
(c) By being permanently attached to what the child is wearing, it prevents the loss of the teething unit and the need to replace it.
(d) This design allows the baby can teethe at any time, without the parent having to relocate, clean, return or otherwise replace with an additional sanitary teething item.
(e) Each time the article of clothing is cleaned, the teething strip is cleaned right along with it, eliminating the need for excess hand-cleaning of teething items.

By permanently attaching a teething strip to children's clothing, this invention has the advantages of allowing easy and consistent access to soothe teething pain while at the same time, limiting the exposure of it being dropped onto unsanitary surfaces, eliminating the need to purchase additional teething items and providing a unique design that enhances any outfit.

Further objects and advantages of the infant clothing and accessories with permanent teething element will become apparent from a consideration of the drawings and ensuing description.

### SUMMARY OF THE INVENTION

The present invention has the advantage of providing easy access to teething items while at the same time preventing the loss of teething unit and limiting the exposure of the teething item being dropped onto unsanitary surfaces.

### DRAWINGS: FIGURES

FIGS. 20 and 22 are a view of an embodiment of the teething strip;

FIGS. 24 and 26 are a view of an alternate embodiment of the teething strip;

FIGS. 28 and 30 are views of more alternate embodiments of the teething strip;

FIGS. 32 and 34 are views of more alternate embodiments of the teething strip;

FIGS. 36 and 38 are views of more alternate embodiments of the teething strip;

FIGS. 40 and 42 are views of more alternate embodiments of the teething strip.

FIG. 44 is a view of another alternate embodiment of the teething strip.

### DRAWINGS: REFERENCE NUMERALS

| | | | |
|---|---|---|---|
| 20 | teething strip at neckline | 22 | teething strip at neckline |
| 24 | teething strip on chest | 26 | teething strip on chest |
| 28 | teething strip on collar | 30 | teething strip on sleeves |
| 32 | teething strip on bib | 34 | teething strip on bib/neckline |
| 36 | teething strip on wrist, | 38 | teething strip on wrist, |
| | fingertips and hand | | fingertips and hand |
| 40 | teething strip on wrist | 42 | teething strip from shoulders |
| 44 | teething strip from headwear | | |

### DETAILED DESCRIPTION

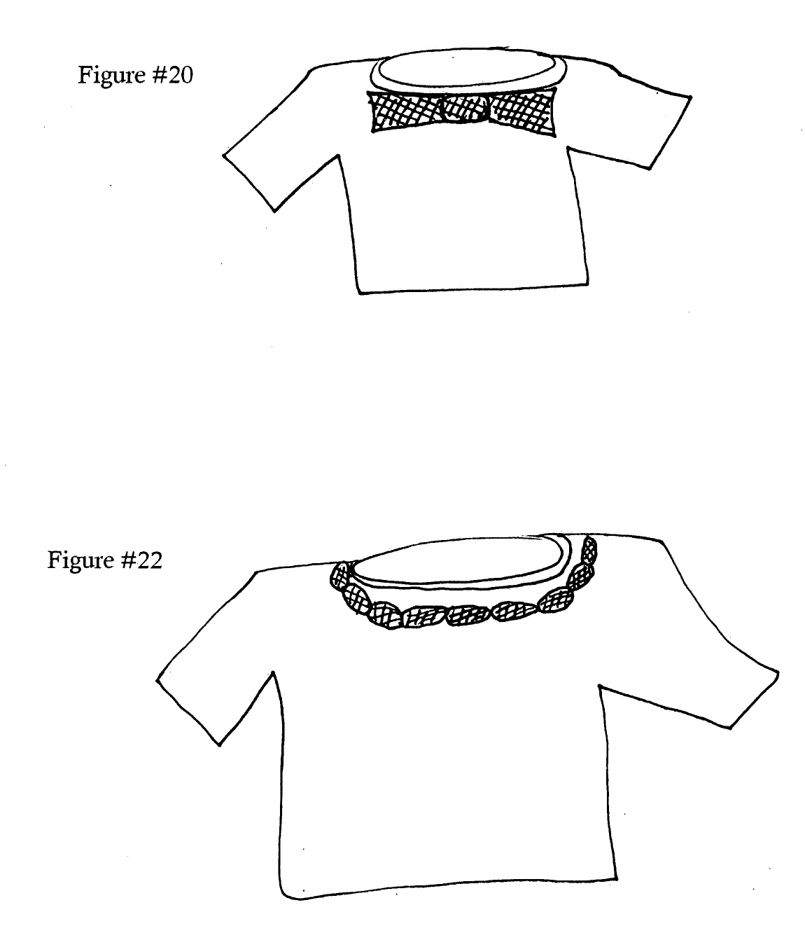

FIGS. 20 and 22 show two separate designs for the teething strip to be attached to an article of clothing or something worn by an individual.

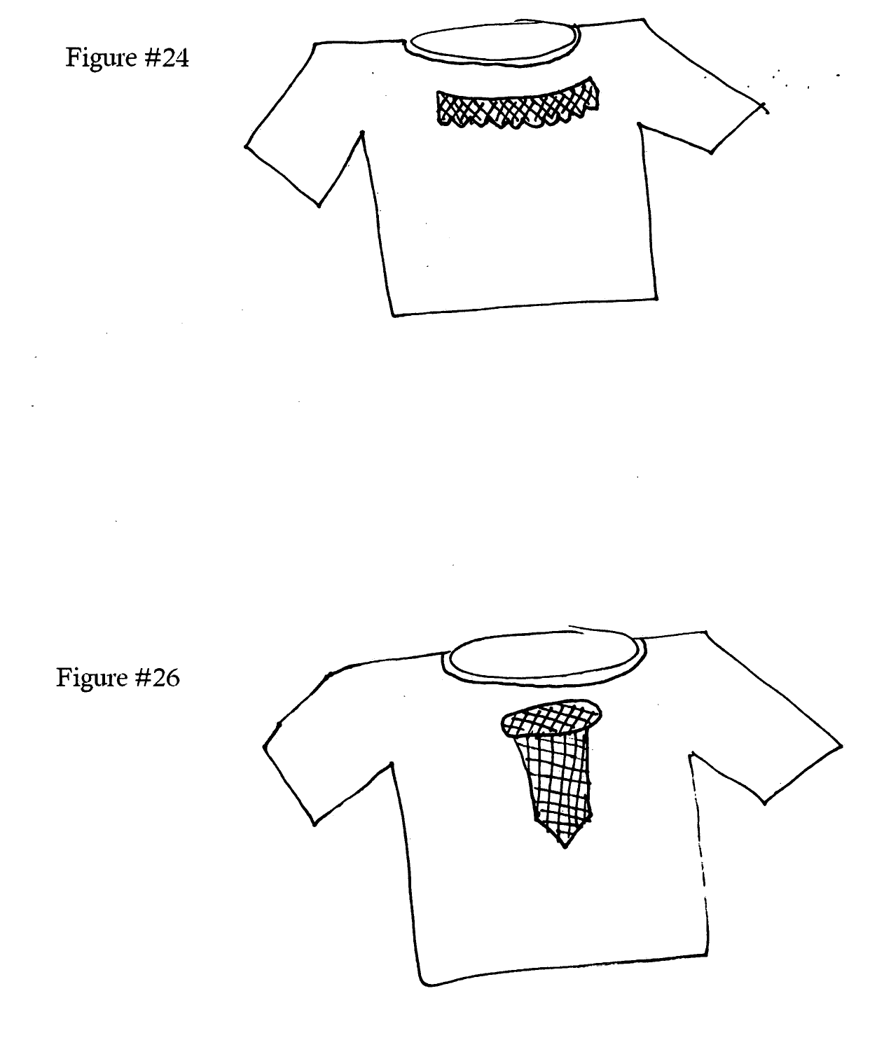

FIGS. 24 and 26 show two separate designs for teething strips to be attached to an article of clothing or something worn by an individual.

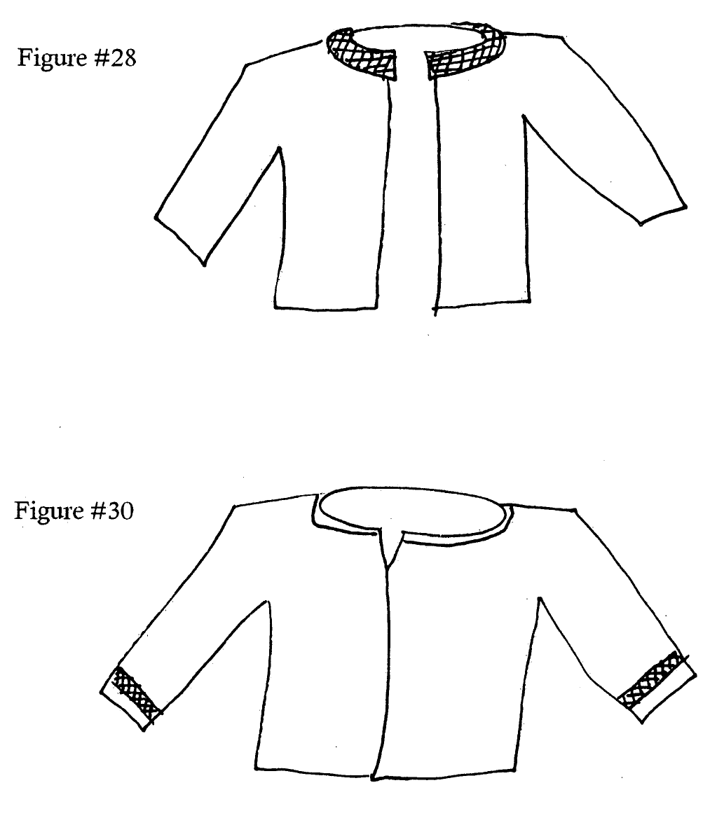

FIG. 28 shows the teething strip attached to the collar of a shirt, pajamas or jacket or to an article of clothing or something worn by an individual.

FIG. 30 shows the teething strip attached to the sleeves of a shirt, pajamas or jacket or to an article of clothing or something worn by an individual.

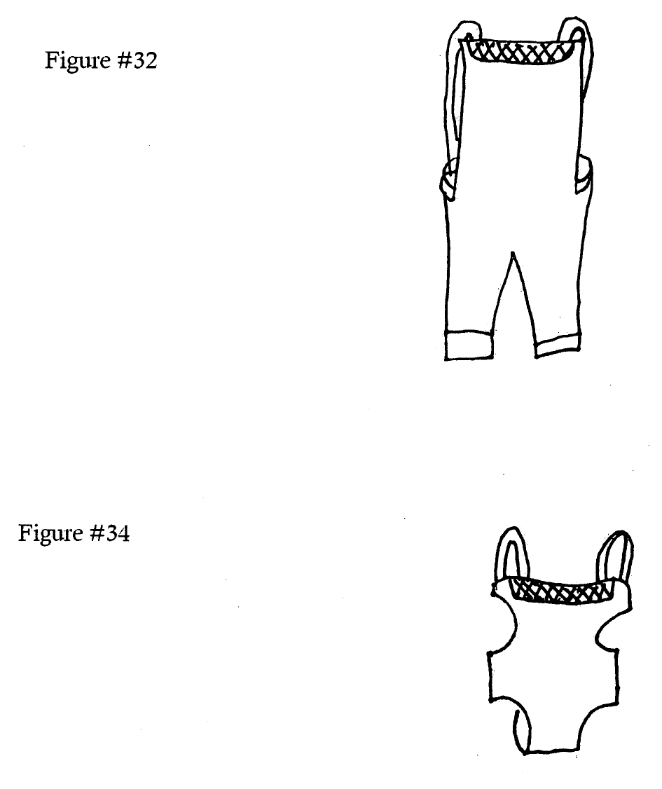

FIGS. 32 and 34 show the teething strip attached across the top of the bib portion of overalls, a bathing suit or baby carrier that an adult uses to carry an infant or to an article of clothing or something worn by an individual.

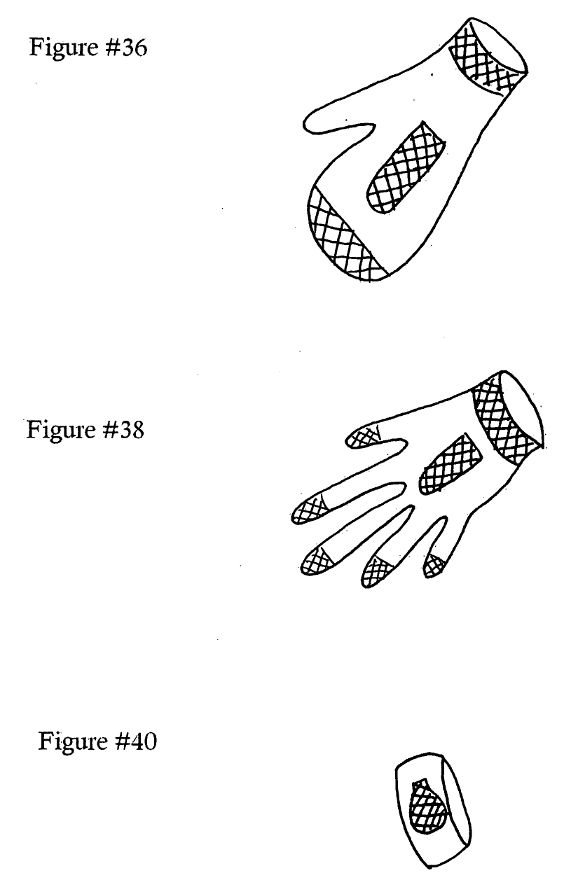

FIGS. 36 and 38 show teething strips attached to the wrist, fingertips and to the back of the hand of mittens and gloves or to an article of clothing or something worn by an individual.

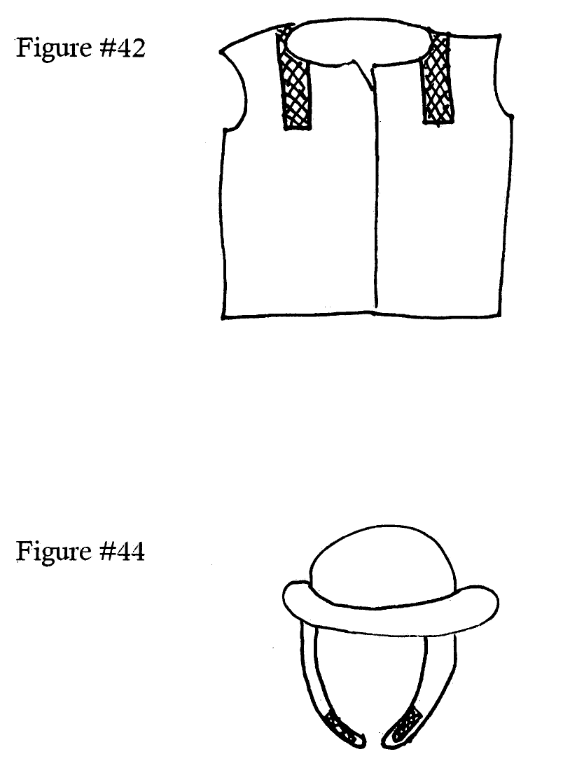

FIG. 40 shows the teething strip attached to a wristband or watchstrap or to an article of clothing or something worn by an individual.

FIG. 42 shows teething strips attached to the shoulders of a vest, which, can also be applied to a shirt, jacket or pajamas or to an article of clothing or something worn by an individual.

FIG. 44 shows teething strips attached to headwear an article of clothing or to something worn by an individual.

### OPERATION OF THE INVENTION

FIGS. 20 and 22 show two separate designs for the teething strip to be attached at the neckline of a shirt. Since being firmly attached only at the top portion of the teething strip, it can easily be flipped upward toward the child's mouth.

FIGS. 24 and 26 show two separate designs for teething strips to be attached to fabric across the chest of a shirt. As each design is firmly attached only at the top of the teething strip, it can easily be flipped upward toward the child's mouth.

FIG. 28 shows the teething strip attached to the collar of a shirt, pajamas or jacket. Since the collar is already flexible, the teething strip can be firmly attached to the collar and allow for the collar to be flipped upward toward the child's mouth.

FIG. 30 shows the teething strip attached to the sleeves of a shirt, pajamas or jacket. Since the teething strip is attached firmly to the sleeve, the child can raise an arm toward the mouth for easy access. This also allows the teething device to be available for indoor or outdoor activity.

FIGS. 32 and 34 show the teething strip attached across the top of the bib portion of overalls, a bathing suit or baby carrier that a parent wears. As each design is firmly attached only at the top of the teething strip, it can easily be flipped upward toward the child's mouth. This allows the teething item to be available while playing in overalls, out swimming, or on a walk being transported in a baby carrier.

FIGS. 36 and 38 show teething strips attached to the wrist, fingertips and to the back of the hand of mittens and gloves. Since the teething strip is attached firmly to the mitten or glove, without having to expose their hands to cold weather when needing to soothe their teething needs.

FIG. 40 shows a teething strip attached to a wristband or toy watch strap. Since the teething strap is firmly attached to the wristband/watch strap, the child can raise a hand toward their mouth for easy access whether they are in water or out.

FIG. 42 shows teething strips attached to the shoulders of a vest. This can also be applied to a shirt, jacket or pajamas. Since the teething strip is attached at the shoulders, the child can easily move the lower portion toward the mouth.

FIG. 44 shows teething strips attached to headwear. Since the teething strip is firmly attached to the straps or sides of a hat, it can easily be moved to reach the child's mouth.

The material and design used on the teething strip may vary, depending on the type of pattern, surface coarseness, firmness or flexibility required.

The present invention provides a simple and effective way for babies to have easy and consistent access to a teething apparatus. The teething strip, usually made of non-toxic plastic including but not limited to corn starch derived plastic, natural rubber, and acrylic, is attached by mechanical means (sewn, snapped, clamped, clipped or riveted) to the baby's clothing or accessories within reach of the baby's mouth. When and if the infant drops the teething element, the element does not fall to the floor or otherwise out of the infant's reach. Thus the baby can teethe at any time, without the parent having to relocate, clean, carry or otherwise replace the teething strip with an additional teething item. Each time the article of clothing is cleaned, the teething strip is cleaned right along with it, eliminating the need for excess hand-cleaning of separate teething items. Since the teething strip can be attached to so many articles of clothing and accessories, this meets the need for the child to have access to convenient teething comfort whether at home in their pajamas, out for a walk in Mom's baby carrier or dressed up for a social event. In addition, this practical application of a teething item provides a unique design that enhances any outfit.

What is claimed is:

1. An infant product, comprising:
an article of clothing; and,
a teething element, permanently attached to the article of clothing.
